(19) United States
(12) Patent Application Publication
(10) Pub. No.: US 2026/0055374 A1
(43) Pub. Date: Feb. 26, 2026

(54) EXPANSION OF HUMAN CELLS AND TISSUES FOR THERAPEUTIC PURPOSES AND METHODS OF USE

(71) Applicant: HOUSEY PHARMACEUTICAL RESEARCH LABORATORIES, L.L.C., Southfield, MI (US)

(72) Inventors: Gerard M. Housey, Southfield, MI (US); Monica E. Balash, Farmington Hills, MI (US); Owen M. Zak, Southfield, MI (US)

(21) Appl. No.: 19/255,463

(22) Filed: Jun. 30, 2025

Related U.S. Application Data
(63) Continuation of application No. PCT/US2023/086579, filed on Dec. 31, 2023.
(60) Provisional application No. 63/436,546, filed on Dec. 31, 2022.

(51) Int. Cl. C12N 5/071; A61K 35/30; A61K 35/39; A61P 3/10; A61P 25/28; C12N 5/0793

(52) U.S. Cl. CPC C12N 5/0676; A61K 35/30; A61K 35/39; A61P 3/10; A61P 25/28; C12N 5/0619

(57) ABSTRACT

The present disclosure provides methods of expanding tissue samples derived from a human being for the purpose of treating certain diseases. The methods include isolation of a small amount of the relevant tissue through biopsy or other methods of tissue acquisition, expanding such tissues ex vivo while retaining natural functioning, and reimplantation of the expanded tissues to alleviating human disease. One example includes ex vivo expansion of human pancreatic islet tissue and reimplantation of the expanded cells into an individual with Type 1 Diabetes for the treatment and long term remission or cure of the disease.

### CROSS-REFERENCE TO RELATED APPLICATIONS

This application is a continuation under 35 U.S.C. 111(a) of International Application No. PCT/US2023/086579, filed Dec. 31, 2023, which claims the benefit of U.S. Provisional Application Ser. No. 63/436,546, filed Dec. 31, 2022, the disclosures of which are incorporated by reference herein in their entireties.

### BACKGROUND

Certain cell types in the body are capable of continuous growth and division. These include skin, various hematopoietic bone marrow precursor cells, and the gut epithelium. For example, it has been estimated that the human erythron, comprised principally of red blood cells (RBCs) and the corresponding bone marrow where RBCs are synthesized, produces more than 100 billion RBCs daily (>2 million cells per second; Sender and Milo, 2021).

On the other hand, certain tissues in the adult human body are comprised of cells that are essentially locked out of the cell division cycle under most circumstances. This is because certain functional cell types in the human body require precise control of cell division in order to maintain the health and well-being of the organism. Endocrine cells are one such example. These cells, which secrete hormones into the bloodstream that are critical for normal physiologic homeostasis, are an example of cells whose division is highly constrained by the organism.

Hormone secreting tissues must be carefully controlled in order to prevent an excessive amount of production of any given hormone. Many endocrine cells synthesize a given hormone both constitutively as well as on a pulsatile, reactive basis in response to changes in the physiology of the organism. When cells constitutively produce a given hormone, careful control of the total number of such cells in the organism is mandatory for maintaining the health of the organism. Should excessive numbers of hormone-producing cells arise as a result of insufficient control of cell division (from sporadic mutation, for example), then the continuous production of a hormone such as thyroid hormone, epinephrine or insulin will lead to an overdose of the corresponding hormone. If left untreated, excessive hormone production will result in serious disease and may be fatal.

Human diseases such as pheochromocytomas, growth-hormone secreting pituitary tumors, thyroid tumors which secrete T3 and T4, and insulin secreting pancreatic tumors known as insulinomas, are representative examples of cellular overgrowths where the corresponding secreted hormone may induce morbidity or even mortality. For example, if left untreated an insulinoma can cause excessive insulin secretion leading to episodes of severe hypoglycemia that may cause convulsions and death, despite the fact that these tumors are generally small, rarely metastatic, and easily cured with surgical removal (Kasper et al. 2015: Harrison's Principles of Internal Medicine).

Due to the aforementioned reasons, long-lived organisms with average life spans of 10 years or more such as human beings and other mammals maintain careful control over cell division for certain classes of cell types within the body. Human cells maintain multiple levels of cell division locking mechanisms to prevent the unintentional overgrowth of certain cell types such as endocrine cells, neurons, and glial cells. These cell division locks, or CDLs, maintain cells in the G₀ state and prevent them from entering the cell cycle. One category of CDLs include certain tumor suppressor proteins, whereas another category includes certain metabolic regulatory proteins. Cell types with potent CDLs include neurons that release potent neurotransmitters such as dopamine, glial cells such as oligodendrocytes, Schwann cells that produce the myelin sheath that insulate and protect neurons, endocrine cells such as insulin secreting beta cells and glucagon secreting alpha cells, and so on.

### SUMMARY

Certain human diseases are characterized by the loss of a specific cell type contained within an important tissue or organ within the body. In certain cases in the adult human being, the cells or tissues that are lost are CDL cells. Examples include Type 1 diabetes, selected cases of Type 2 diabetes, Parkinson's disease, Waterhouse-Friderichsen Syndrome, Multiple Sclerosis, and others (Kasper et al, 2015; Gholamzad et al., 2019; Buzzard et al., 2017; Harris et al., 2020; Harris et al., 2020)

In some cases it is desirable to treat the underlying disease with replacement of the lost tissue and relevant cells, a process that has been referred to in broad terms as regenerative medicine. A key challenge in doing so, however, is that it is difficult to stimulate many tissues of the body (and the cell types contained in these tissue) to grow in vivo (inside the body) or ex vivo (outside the body).

Skin is one organ that can effectively be propagated outside the body, and remarkable treatments of human skin disorders such as with burn victims and with patients having rare, life-threatening genetic mutations in skin cell development have been achieved. In one case study, a 7-year old with life-threatening junctional epidermolysis bullosa was effectively treated by transducing human primary keratinocytes derived from the patient with a Moloney Murine Leukemia virus promoter construct harboring an intact, wild type version of the human LAMB3 cDNA (Hirsch et al., 2017). This was a remarkable advance, notwithstanding the fact that human keratinocytes are easily cultured ex vivo.

Other human tissue types are more difficult to culture ex vivo, such as cardiac myocytes. This has led investigators to turn to stem cell-based approaches. The seminal work by Yamanaka and colleagues in Japan demonstrated that mature, differentiated cells may be reprogrammed to an embryonic-like state and then re-differentiated into a variety of mature cell types with selected properties that are similar to their mature wild type counterparts (Takahashi and Yamanaka, 2006; Yamanaka, 2020). Over the last two decades, substantial research has been done in the induced pluripotent stem cell (iPSC) field in an attempt to realize the potential of regenerative medicine, with mixed results as discussed below. What has become clear is that the process of reprogramming stem cells appears to resemble the developmental pathways that occur during human embryogenesis, but with key differences that are not yet understood which may impact the long term safety and efficacy of therapeutic procedures based upon their use.

For example, in an effort to generate sufficient numbers of cardiac muscle cells (myocytes) for the purpose of replacing damaged cardiac tissue that results from a heart attack, researchers turned to a stem-cell based approach to generate sufficient amounts of myocyte-like cells. iPSCs were utilized to generate myocyte-like cells in abundance which were then utilized in an attempt to repair the hypoxia-induced scar tissue formation that results following a myocardial infarction. When reintroduced in the heart of a primate, the resulting stem cell-derived myocytes had difficulty functioning normally. The stem cell-derived myocytes did not contract in unison with existing cardiac myocytes and were unable to adapt to the normal electrical rhythm. Abnormalities in the electrocardiograms (ECG) of reimplanted primates were evident which led to the development of life-threatening arrythmias (EbioMedicine, 2018).

Both embryonic stem cells as well as induced pluripotent stem cells may be differentiated into a variety of cell types seemingly identical to the corresponding fully differentiated human cell type. When gene expression analyses are performed using cells derived from fully differentiated adult human cells derived from a specific tissue such as cardiac myocytes or pancreatic insulin-producing beta cells, and similar cell types are produced from stem cells differentiated in the laboratory, numerous gene expression differences become evident (Yamanaka, S., 2020). This has called into question the long-term safety of stem-cell based therapies that are intended for reintroduction into humans for therapeutic purposes (Kushner et al., 2014). Indeed, there are numerous examples of tumors arising after introduction of stem-cell derived tissues into experimental animals. Teratomas, which are undifferentiated tumors that can be aggressive, have been shown to form from all three primary embryonic tissue layers in mammals (ectoderm, mesoderm, and endoderm). This represents a serious long term safety risk associated with the potential clinical use of iPSCs in human beings (Blum et al., 2009; Cunningham et al. 2012; Fong et al., 2010; Kum et al., 2012; Lee et al., 2009; Mutter et al., 1987; Prokhorova et al., 2009).

The aforementioned problems and safety concerns associated with stem cell use would be circumvented if a technology existed that could induce a controlled release of the cell division locking mechanisms that prevent certain adult tissues and cell types such as human beta cells from undergoing substantial growth and division. This disclosure provides, for the first time, the ability to select a therapeutically relevant, fully differentiated, non-dividing cell or group of cells and temporarily release them from the cell division lock in order to allow for a period of controlled cell division until such time as a therapeutically sufficient amount of the cells or tissue have been created. This is accomplished by inducing a temporary steady state thermodynamic alteration of energy utilization in the cell to enable transition into the cell cycle without resorting to the use of recombinant DNA methodologies or techniques such as CRISPR/CAS-9 genomic editing methods. The latter are to be avoided in order to maintain the genomic integrity of an otherwise healthy human cell type that is intended for reimplantation and is only lacking in relative abundance in the body. Thus, for the purposes of patient safety it is preferable to avoid the use of recombinant DNA and genome editing methods, whereas temporal modulation of RNA expression levels through the use of gene silencing methods such as small interfering RNAs (siRNAs) is both useful and a preferred embodiment.

The essence of the disclosure involves the temporal disengagement of the normal regulation of cell division locks that limit the ability to grow certain human tissue and cell types ex vivo. By temporarily removing, in a controlled, reversible manner, the cell division locks (CDLs) that prevent critically important tissue-specific cells such as endocrine cells or neurons from entering the cell cycle, the technology described herein renders it possible to expand such cell types ex vivo while maintaining their primary phenotypic characteristics as mature, fully differentiated cells. This methodology is fundamentally distinct from existing approaches involving the use of embryonic stem cells (ES cells) or induced pluripotent stem cells (iPSCs) where cells are first grown to sufficient amounts in a dedifferentiated state and then subsequently redifferentiated into a cell type with properties approximating the therapeutically desired cell type. FIG. 1 provides a diagram to outline the difference between the current approaches to stem-cell based therapies and the disclosure provided herein.

Thus, this disclosure enables the skilled investigator to expand ex vivo a selected cell type to a therapeutically sufficient amount for the purpose of enabling the future reimplantation of the cell type into a human being while avoiding the need to use a stem cell-based approach to achieve sufficient cell growth. This methodology avoids the safety, gene expression variability and tumorigenicity concerns associated with stem cell use (Ohnishi et al, 2014; Yasuda et al, 2018).

Terms used herein will be understood to take on their ordinary meaning in the relevant art unless specified otherwise. Several terms used herein and their meanings are set forth below.

As used herein, the term “tissue,” is a group or layer of similarly specialized cells which together perform certain special functions.

As used herein, the terms “compound” and “substance” are used interchangeably, and both terms refer to chemical agents and biological agents.

As used herein, the term “chemical agent” refers to substances that have a molecular weight up to, but not including, 2000 atomic mass units (Daltons). Such substances are sometimes referred to as “small molecules.”

As used herein, “biological agent” refers to molecules that include proteins and polynucleotides, and have molecular weights equal to or greater than 2000 atomic mass units (“amu”or “Daltons”), but not to exceed 990,000 amu.

As used herein, the terms “activator”and “agonist”are used interchangeably.

As used herein, the terms “inhibitor”and “antagonist”are used interchangeably.

An “inhibitor of expression” is meant to include a compound that inhibits the expression of the gene by any mechanism, including interference with the production of functional mRNA or enhancing degradation of mRNA.

For this disclosure, to state that a substance “inhibit(s)” means the substance can bind to and reduce activity of a protein in a cell, a tissue, the blood, or presence in the body; the substance can reduce or eliminate functioning of a protein; the substance can reduce the amount or level of a protein; and/or the substance can reduce the expression or production of a protein.

Unless explicitly stated otherwise, an “inhibitor” and an “antagonist” are synonymous. The inhibition by an inhibitor may be partial or complete. The terms “bind(s),” “binding,” and “binds to” have their ordinary meanings in the field of biochemistry in terms of describing the interaction between two substances (e.g., enzyme-substrate, protein-DNA, receptor-ligand etc.). As used herein, the term “binds to” is synonymous with “interacts with” in the context of discussing the relationship between a substance and its corresponding target protein or nucleic acid.

As used herein, the term “antibody” refers to a protein or immunoglobulin produced in response to an antigen and can “specifically bind” the antigen. An antibody that “specifically binds” an antigen is one that interacts only with the epitope of the antigen that induced the synthesis of the antibody, or interacts with a structurally related epitope. An antibody that “specifically binds” to an epitope will, under the appropriate conditions, interact with the epitope even in the presence of a diversity of potential binding targets.

As used herein, the term “antigen” refers to the protein or peptide target having the epitope to which an antibody specifically binds.

As used herein, the term “fragment” refers to a portion of a polypeptide or polynucleotide. In one embodiment, a fragment retains the activity of the polypeptide or polynucleotide.

According to the present disclosure, a therapeutically effective amount of one or more compounds/substances that up regulate/activate, or down modulate/inhibit, for instance, the function or level of expression of a viability target (VT) protein or a protein that degrades a VT, respectively, is administered to a cell obtained from a mammal, or a mammal in need thereof. The term “mammal” as used herein is intended to include, but is not limited to, humans, laboratory animals, domestic pets, and farm animals.

The phrase “effective amount” as used herein means that amount of a compound, material, cell, group of cells, or composition comprising a compound of the present disclosure which is effective for producing some desired effect in at least a sub-population of cells (e.g., pancreatic cells) in an animal, such as increasing expression of the viability target, increasing the amount of the viability target, and/or reducing the activity of proteins or nucleic acid sequences that inhibit the viability target.

The phrase “therapeutically-effective amount” as used herein means that amount of a compound, material, cell, group of cells, or composition comprising a compound of the present disclosure which is effective for producing some desired therapeutic effect in at least a sub-population of cells (e.g., pancreatic cells) in an animal at a reasonable benefit/risk ratio applicable to any medical treatment, e.g. reasonable side effects applicable to any medical treatment.

Unless otherwise specified, “a,” “an,” “the,” and “at least one” are used interchangeably and mean one or more than one.

Conditions that are “suitable” for an event to occur, or “suitable” conditions are conditions that do not prevent such events from occurring. Thus, these conditions permit, enhance, facilitate, and/or are conducive to the event.

As used herein, “providing” in the context of a composition, a cell, a group of cells, an antibody, a nucleic acid, or a small molecule means making the composition, antibody, nucleic acid, or small molecule, purchasing the composition, antibody, nucleic acid, or small molecule, or otherwise obtaining the composition, antibody, nucleic acid, or small molecule.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example: 1, 2, 2.7, 3, 4, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

### BRIEF DESCRIPTION OF THE FIGURES

The following detailed description of illustrative embodiments of the present disclosure may be best understood when read in conjunction with the following drawings.

FIG. 1 shows a schematic diagram of the differences between expanding cell mass using stem cells versus fully differentiated mature cells. The stem cell approach involves a three step process: (1) de-differentiating a mature cell into an induced pluripotent stem cell (iPSC), whereby the phenotype of the mature cell is lost, (2) propagating the iPSCs until a sufficient amount are available, and (3) redifferentiating the iPSCs into structurally and functionally similar, but non-identical mature, differentiated cells. The novel method involves retention of the fully differentiated phenotype, with induction of the cell into the cell cycle in a temporary, reversible manner.

FIG. 2 shows enhancement of viability of 32D cells expressing insulin receptor and insulin receptor substrate-2 (IRS-2) following treatment with insulin (INS) or an activator of IRS (HP-508). 32DIR-IRS-2 cells treated with the IRS Activator exhibit approximately equal growth to INS-treated cells but have enhanced viability after 72 hours of culture as compared to cells treated with INS (Housey and Balash, 2018; Housey and Balash, 2023). Note the numerous cytoplasmic inclusions in the INS-treated cells, reflecting increased cellular stress and reduced viability. In contrast, the IRS Activator treated cells exhibit multiple regions of intact cell morphology (circled). INS-treated cells generally exhibit viability values of 60% or lower, whereas IRS Activator-treated cells can have viabilities exceeding 90% after 72 hours (data not shown). Viability was determined using Trypan Blue exclusion with an automated cell counter.

FIGS. 3A and 3B show growth curves of human islet tissue maintained in ex vivo culture in the presence of an IRS Activator and a growth-enhancing substance. Growth of human islet tissue containing beta cells in the presence of an IRS-2 activator (HP-508) together with recurring treatment with various siRNA's as indicated. Tissue growth was determined by digital imaging with a Zeiss CD-7 automated microscope. Total tissue surface area was computed using ZEN software. Under these conditions, human islet tissues under 300 microns in long dimension generally develop into tissue clumps ranging from 15-30 microns in vertical height (thickness). TP53, growth in presence of siRNA (see FIG. 4) that inhibits expression of the human tumor suppressor protein P53; STK4, growth in presence of siRNA (see FIG. 4) that inhibits expression of the human serine/threonine kinase 4 gene; VGLL4, growth in presence of siRNA (see FIG. 4) that inhibits expression of the human vestigial like family member 4 gene; TSC-1, growth in presence of siRNA (see FIG. 4) that inhibits expression of the human tuberous sclerosis 1 gene.

FIGS. 4A and 4B show examples of the siRNAs synthesized and tested. Each siRNA is shown in two formats. The top shows each strand of the duplex 5′ to 3′ and identifies ribonucleotides with “r” before the nucleotide A, U, G, or C. Those nucleotides without “r” before the nucleotide A, T, G, or C identifies deoxyribonucleotides. The bottom shows the two strands of the duplex hybridized.

FIG. 5 shows insulin production by cultured human islets. Islets continue to produce Insulin throughout the growth period.

FIG. 6 shows a representative human islet-like tissue mass, approximately 1,000 micrometers (μm)×400 μm×170 μm, propagated from a small group of human donor cells (<10) using an IRS activator. The representative human islet tissue mass shown was maintained in culture for more than one year. Abbreviations are as follows: i.t.—islet tissue; v.—vessel outgrowths. Note that the tissue mass exceeds 1,000 microns in long dimension. Vascular-like vessel outgrowths are visible coursing throughout the tissue mass. These structures appear during the middle and later stages of growth. Addition of a growth enhancer (also referred to herein as a cell division enhancer) as shown in FIG. 3 can reduce the total time for growth of a tissue mass sufficient to support reimplantation to as little as 120-180 days.

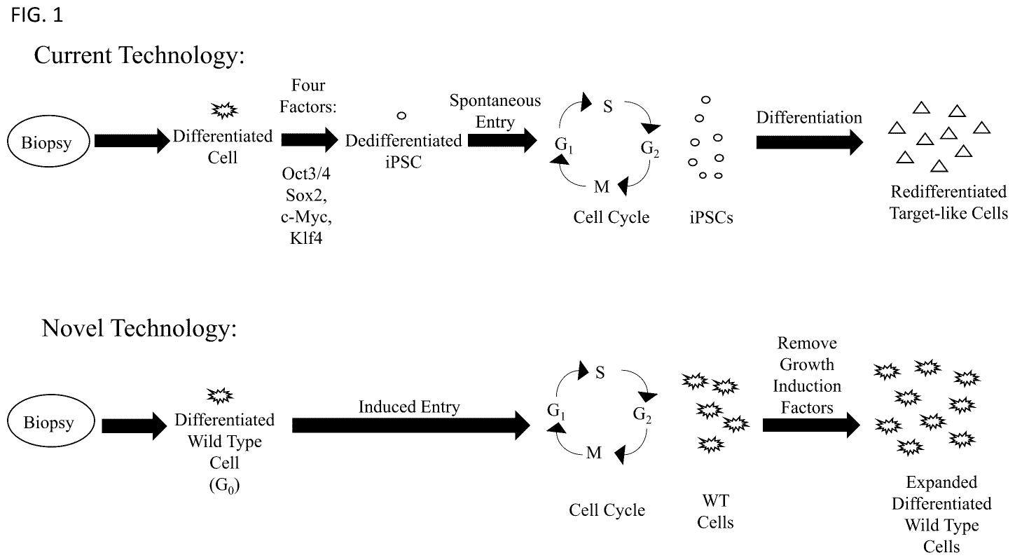

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION

The present disclosure includes a method for the ex vivo expansion of cells from a patient. The method can include providing a tissue sample or similarly specialized cells (cells of interest) from a subject, such as a tissue sample that was obtained by biopsy or an enriched or purified population of cells of interest. The tissue can include similarly specialized cells which together perform a certain specific function. The tissue can be from an individual with a disease, where pathology of the disease includes loss of the tissue. Examples of such diseases include but are not limited to Type 1 diabetes (T1D) and Parkinson's Disease. In one embodiment, the disease is T1D and the subject has a relative or absolute deficiency of insulin. The similarly specialized cells are the cells that are to be expanded ex vivo. When the disease is T1D, examples of tissues and similarly specialized cells include but are not limited to pancreatic tissue, islets of Langerhans, and beta cells. When the disease is Parkinson's Disease, examples of tissues and similarly specialized cells include but are not limited to substantia nigra tissue and dopaminergic neurons.

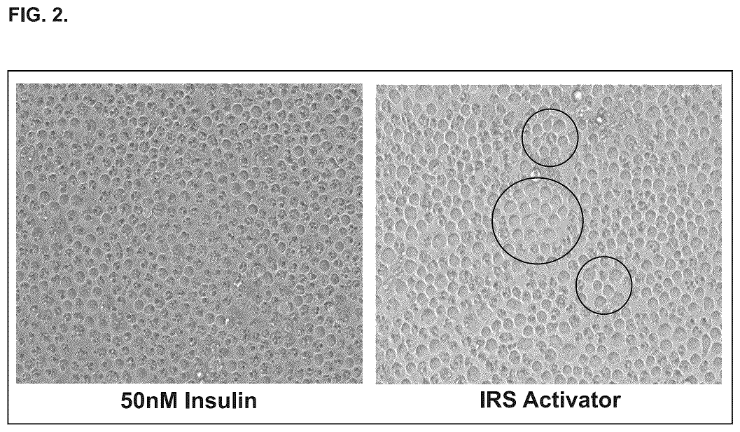

The tissue with the desired cells of interest is established in culture. In one embodiment, this can further include establishing the cells, e.g., tissue sample and/or cells of interest, in culture which can be accomplished with a known rich culture medium such as Dulbecco's Modified Eagle's Medium supplemented with 10% human serum. Other types of media may be utilized (Yao and Asayama, 2017). If necessary, a higher percentage of serum may be used. If the relevant cell type is viable in ex vivo culture, then identifying a Viability Target and identifying a compound that activates, stabilizes, or reduces degradation of the Viability Target is not necessary. The terms viable and viability refer to maintaining the cell type in culture without appreciable loss of the total number of cells, although no net cell growth is necessary. FIG. 2 shows a representative example of the effect of compound HP-508 on human beta cell viability in culture.

Expansion of the cells, e.g., tissue sample and/or cells of interest includes culturing in the presence of a compound that activates a target protein whose cellular function is essential for maintaining cellular viability when the desired cell type is grown in culture in the laboratory. This target protein or nucleic acid sequence is referred to as a Viability Target (“VT”). An example of Viability Targets for some cells is an IRS-2 protein. Cells expressing an IRS-2 protein include islets of Langerhans cells, e.g., beta cells, and substantia nigra tissue, e.g., dopaminergic neurons. In other embodiments, a Viability Target for a particular tissue or cell can be identified by selecting a target protein or nucleic acid sequence (such as an miRNA) from the human genome (e.g., the REF genbank). This can be accomplished using a variety of standard methodologies, but one of the most common is through the use of targeted gene knockout studies in mice or other primates. The majority of cell types will not require the use of a Viability Target, as it is well established that most human and other mammalian cell types remain viable in ex vivo culture even if no cell growth/expansion is obtained. In the rare cases where, as with human beta cells, explanted islets tissue containing viable beta cells do not remain viable in culture indefinitely, then a suitable Viability Target may be identified through the use of systematic gene knockout studies using individual genes that are expressed in the cell type of interest. Gene expression analysis as well as the generation of knockout mice is standard in the art. In such cases, identification of a suitable Viability Target is determined by the loss of the corresponding cell type of interest during embryogenesis or in the early postnatal period of the knockout mouse, where progressive loss of the relevant cell type will occur due to the absence of the corresponding gene encoding the VT protein. (Withers et al., 1998; Withers et al, 1999; Hennige et al., 2003). Example 1 provides such an approach.

In those embodiments where a Viability Target is to be identified, the method further includes selecting or designing a compound known to activate, stabilize or reduce the degradation of the VT. Such a compound is referred to herein as a Viability Target Enhancer (“VTE”). If a compound is already known to activate the VT, the compound may be used. Alternatively, if no such compound exists, one may create such a compound, for example, by using an anti-sense oligonucleotide or a small interfering ribonucleic acid (“siRNA”) targeting a gene known to result in degradation of the VT. The net effect of such treatment is to increase the relative intracellular abundance of the VT. A VTE that activates, stabilizes, or reduce the degradation of the Viability Target IRS-2 protein includes IRS Activators. A nonlimiting example of an IRS Activator is HP-508 (Housey and Balash, 2018; Housey and Balash, 2023).

Expansion of the cells, e.g., tissue sample and/or cells of interest includes culturing in the presence of a substance that enhances cell division. In one embodiment, the substance is one that reduces the expression of a cell division locking (CDL) protein. Reducing expression of a CDL protein leads to the initiation and maintenance of cell growth. Use of a substance that enhances cell division can result in minimizing the amount of time that cells are grown ex vivo prior to reimplantation into a patient. Keeping the ex vivo culture time as short as possible serves to maintain the phenotypic characteristic(s) of the selected cell type (hormone secretion, neurotransmitter release, etc.) that are necessary to obtain a therapeutically effective cell population for reimplantation. Suitable CDL proteins include, but are not limited to proteins that control metabolic state and proteins that prevent tumor formation (tumor suppressors). Specific examples include, but are not limited to, siRNAs that are synthesized and tested individually for the ability to enhance growth of the cells of interest, wherein specific siRNAs are designed to target proteins encoded by one of the following genes: activin gene (all variants), myosin (all variants), ANX7, APC, ARF, ATM, ATR, BCL2, BECLIN1, BIM, BLM, BMPR, BRCA1, BRCA2, BUB3, α-Catenin, CBFA2/AML1/RUNX1, CDH1 (E-CAD; E-Cadherin), CDKN1A, CDKN1B, CDKN2A, CDKN2C, CHK1, EXT1, EXT2, FBXW7 (CDC4), FEN1, FHIT, FST, H2AX, HIPK2, HRPT2, INPP4B, Integrin, LIG4, LKB1, MAD2, MEN1, MEN2, MKNK2, MLH1, MSH2, MSH6, MUTYH, NBS1, NF1, NF2, NKX3.1 (NKX3A), P15, P53, PLK4, PMS1, PMS2, PTC, PTCH, PTEN, PTPN1, RB, RB1, RECQL4, Ribosomal Protein Gene (L35, L37A, RPS19, S8), RNASEL, SDH, SMAD2, SMAD3, SMAD4/DPC4, STK4, SU(FU), TEAD1,TGFH, TGFBR, TSC-1, TSC-2, VGLL4, VHL, WRN, WT1, and TP53. Specific examples include TP53, STK4, VGLL4, TSC-1, CDKNIB, and CDKN2A (Macleod, 2000; Payne and Kemp, 2005). Optionally, the cells of interest can be used in a screen to identify siRNAs or RNA sequences or RNA analogues capable of reducing the expression of a CDL protein. This will enable the cells of interest to transiently re-enter the cell cycle for a sufficient time period to expand the population to reach a therapeutically sufficient cell mass.

The skilled person will recognize that there are a variety of methods known in the art that can be used to reduce the expression of the CDL protein. Such methods include but are not limited to the following.

Gene Silencing using short interfering RNA (siRNA) and related approaches using RNA interference (RNAi) are a preferred embodiment (Housey and Balash, 2019). It is now well-established that RNAi play an important role in post-transcriptional gene silencing through molecules such as siRNAs. siRNAs are about 19-22 nucleotide (nt) duplex RNA (dsRNA) molecules capable of reducing or silencing the translation of messenger RNAs (mRNAs) in a sequence specific fashion (Walton et al., 2010; Sibley et al., 2010).

Polynucleotides can be used to reduce expression of specific genes. Such inhibitory polynucleotides include RNA interference (RNAi), mediated by double-stranded small interfering RNA (siRNA), which silences a gene with a high degree of specificity. A siRNA includes a sequence that is complementary to a protein coding messenger RNA (mRNA) and causes the degradation of the mRNA. One of ordinary skill in the art can design and synthesize siRNA molecules that are able to reduce the expression of a CDL protein. siRNA molecules for reducing expression of CDL proteins are also commercially available (e.g., Dharmacon, Lafayette, Colo.). Automated synthesis of nucleic acids is well established, and includes modifications at numerous positions on the nucleoside and ribose/deoxyribose ring systems (Sibley et al., 2010; Walton et al., 2010).

Another type of inhibitory nucleotide includes antisense RNA, single stranded RNA complementary to a protein coding mRNA with which it hybridizes, and thereby blocks its translation into protein. A siRNA used in the methods herein has the ability to reduce expression of a CDL protein. RNA interference methods represent a useful approach for molecularly targeted therapy. Thus, in another embodiment, siRNAs or another RNAi methodology is utilized. siRNAs are synthesized and tested for their ability to reduce the expression of a CDL protein in a therapeutically effective manner in a mammal. Oligonucleotide synthetic methods of manufacturing siRNAs are well established. No limitation is intended with respect to the type of RNAi that may be utilized to reduce CDL protein levels in a mammal, including RNA-DNA chimeras, tandem hairpin RNAs, tandem siRNAs, tRNA-shRNAs, and the like (Sibley et al., 2010).

In one embodiment, a polynucleotide useful herein includes a double stranded RNA (dsRNA) polynucleotide. The sequence of a polynucleotide includes one strand, referred to herein as the sense strand, of 16 to 30 nucleotides, for instance, 16, 17, 18, 19, 20, 21, 22, 23, 24, 25, 26, 27, 28, 29, or 30 nucleotides. The sense strand is substantially identical, preferably, identical, to a target mRNA, e.g., an mRNA that encodes a CDL protein. As used herein, the term “identical” means the nucleotide sequence of the sense strand has the same nucleotide sequence as a portion of the target mRNA. As used herein, the term “substantially identical” means the sequence of the sense strand differs from the sequence of a target mRNA at 1, 2, or 3 nucleotides, preferably 1 nucleotide, and the remaining nucleotides are identical to the sequence of the mRNA. These 1, 2, or 3 nucleotides of the sense strand are referred to as non-complementary nucleotides. When a polynucleotide includes a sense strand that is substantially identical to a target mRNA, the 1, 2, or 3 non-complementary nucleotides are preferably located in the middle of the sense strand. For instance, if the sense strand is 21 nucleotides in length, the non-complementary nucleotides are typically at nucleotides 9, 10, 11, or 12, preferably nucleotides 10 or 11. The other strand of a dsRNA polynucleotide, referred to herein as the anti-sense strand, is complementary to the sense strand.

The sense and anti-sense strands of a dsRNA polynucleotide may also be covalently attached, typically by a spacer made up of nucleotides. Such a polynucleotide is often referred to in the art as a short hairpin RNA (shRNA). Upon base pairing of the sense and anti-sense strands, the spacer region forms a loop. The number of nucleotides making up the loop can vary, and loops between 3 and 23 nucleotides have been reported (Sui et al., Proc. Nat'l. Acad. Sci. USA, 99, 5515-5520 (2002), and Jacque et al., Nature, 418, 435-438 (2002)).

In one embodiment, a polynucleotide useful herein includes single stranded RNA (ssRNA) polynucleotides. The sequence of a polynucleotide includes one strand, referred to herein as the anti-sense strand, of at least 16 nucleotides. The anti-sense strand is substantially complementary, preferably, complementary, to a target mRNA, e.g., an mRNA that encodes a CDL protein. In one embodiment, a polynucleotide for decreasing expression of a coding region in a cell includes substantially all of a coding region, or in some cases, an entire coding region. An antisense strand is substantially complementary, preferably, complementary, to a target coding region or a target mRNA. As used herein, the term “substantially complementary” means that at least 1, 2, or 3 of the nucleotides of the antisense strand are not complementary to a nucleotide sequence of a target mRNA.

Polynucleotides of the present disclosure are preferably biologically active. A biologically active polynucleotide causes the post-transcriptional inhibition of expression, also referred to as silencing, of a target coding region. Without intending to be limited by theory, after introduction into a cell a polynucleotide of the present invention will hybridize with a target mRNA and signal cellular endonucleases to cleave the target mRNA. The result is the inhibition of expression of the polypeptide encoded by the mRNA. Whether the expression of a target coding region is inhibited can be determined by, for instance, measuring a decrease in the amount of the target mRNA in the cell, measuring a decrease in the amount of polypeptide encoded by the mRNA, or by measuring a decrease in the activity of the polypeptide encoded by the mRNA.

A polynucleotide of the present disclosure may include additional nucleotides. For instance, with respect to the sense strand, the 5′ end, the 3′ end, or both ends can include additional nucleotides, provided the additional nucleotides are identical to the appropriate target mRNA and the overall length of the sense strand is not greater than 30 nucleotides.

A polynucleotide may be modified. Such modifications can be useful to increase stability of the polynucleotide in certain environments. Modifications can include a nucleic acid sugar, base, or backbone, or any combination thereof. The modifications can be synthetic, naturally occurring, or non-naturally occurring. A polynucleotide can include modifications at one or more of the nucleic acids present in the polynucleotide. Examples of backbone modifications include, but are not limited to, phosphonoacetates, thiophosphonoacetates, phosphorothioates, phosphorodithioates, phosphoramidates, methyl phosphonates, chiral-methyl phosphonates, 2-O-methyl ribonucleotides, and peptide-nucleic acids. Examples of nucleic acid base modifications include, but are not limited to, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), or propyne modifications. Examples of nucleic acid sugar modifications include, but are not limited to, 2′-sugar modification, e.g., 2′-O-methyl nucleotides, 2′-deoxy-2′-fluoro nucleotides, 2′-deoxy-2′-fluoroarabino, 2′-O-methoxyethyl nucleotides, 2′-O-trifluoromethyl nucleotides, 2′-O-ethyl-trifluoromethoxy nucleotides, 2′-O-difluoromethoxy-ethoxy nucleotides, or 2′-deoxy nucleotides. Polynucleotides can be obtained commercially synthesized to include such modifications (for instance, Dharmacon Inc., Lafayette, Colo.).

The skilled person will recognize that the method can include confirming that the tissue and/or cells of interest maintain the appropriate phenotypic characteristic(s) of the selected cell type (hormone secretion, neurotransmitter release, etc.) that are necessary to obtain a therapeutically effective cell population for reimplantation. For instance, when the tissue and/or cells of interest originate from pancreatic tissue, e.g., islets of Langerhans, continued production of insulin is desirable, and when the tissue and/or cells of interest originate from substantia nigra tissue, e.g., dopaminergic neurons, continued production of dopamine is desirable.

Once a therapeutically effective amount of cells/tissue is obtained, the cells are reimplanted into the appropriate site within the body. For example, in the case of beta cells for the treatment of diabetes, cells may be infused via the portal vein directly into the liver (McEachron et al., 2018). In the case of dopamine-secreting neurons for the treatment of Parkinson's disease, cells may be reimplanted into the brain by utilizing standard neurosurgical procedures via real-time imaging to guide catheter placement into the substantia nigra, after which the cells are infused in an isotonic buffer solution. The number of cells reimplanted can vary depending upon the status of the disease and tissue loss in the patient. In some embodiments, at least 100,000 cells, at least 200,000 cells, at least 300,000 cells, at least 400,000 cells, at least 500,000 cells, or at least 600,000 cells are administered. In some embodiments, no greater than 1,000,000,000,000 cells are administered.

Master stocks of the tissue and/or cells of interest may be expanded and preserved in frozen storage for future use in the event that years or decades later the disease recurs and reimplantation of additional functional tissue would be therapeutically effective. Through this approach, a durable long-term remission or functional cure of certain diseases such as T1D and Parkinson's Disease is achieved.

Optionally, the methods can further include monitoring the status of the reimplanted tissue. When the reimplanted tissue includes beta cells, the method can further include determining if the conditions associated with T1D are decreased, such as whether blood glucose control is restored in the subject. When the reimplanted tissue includes dopaminergic neurons, the method can further include determining if the conditions associated with Parkinson's Disease are decreased, such as whether dopamine production is increased in the subject. Alternatively, increased levels of brain-derived neurotrophic factor can also be monitored (see Example 2 below).

Type 1 Diabetes (T1D, previously known as juvenile-onset diabetes) results when a patient's immune system mistakenly attacks the beta cell population within the islets of Langerhans in the pancreas. Thus, T1D is an autoimmune disease. The current standard of care for T1D revolves around continuous daily administration of insulin, of which various types have been developed and are available.

Numerous cellular approaches to treat T1D using islets derived from various sources such as bovine, porcine, and human cadaveric donors have been attempted with minimal success. The longest lasting approach has been to transplant human islets isolated from cadaveric donors. Since these donors are not genetically identical to the recipient, the allogeneic transplant recipient must continuously take immunosuppressive drugs to reduce the likelihood of immune-mediated transplant rejection. Such transplants, if successful, generally only last for a period of three to five years, after which time the transplanted tissue ceases to function and the recipient either reverts to the use of daily insulin once again or must undergo a second transplant from another allogeneic donor.

Serious, life-threatening limitations of allogeneic transplantation have resulted in the FDA determining that allogeneic transplantation in all forms is an experimental procedure. A detailed discussion of the potential side effects and adverse events associated with allogeneic transplantation can be found on the National Institute of Diabetes, Digestive Disorders and Kidney Diseases (NIDDK) website (see Pancreatic Islet Transplantation: https://www.niddk.nih.gov/health-information/diabetes/overview/insulin-medicines-treatments/pancreatic-islet-transplantation). As described therein, the most serious side effects and adverse events associated with allogeneic transplantation pertain to the need for chronic immunosuppression in order to avoid graft-vs-host disease as well as immunorejection by the recipient resulting in loss of function of the transplanted donor islets. Chronic immunosuppression is also required even if the donor islets are derived from induced pluripotent stem cells rather than from the pancreas of a cadaveric donor. The recipient may develop antibodies against the donor cells that will complicate the effort to locate an appropriate organ donor if another transplant is needed in the future, which is most often the case.

Most importantly, the chronic use of immunosuppressants, which are necessary to prevent the recipient from destroying the donor islets irrespective whether the source of such islets is derived from iPSCs or a cadaveric donor, lead to many serious side effects. According to the NIDDK website, potential side effects resulting from the chronic use of immunosuppressive medications include an increased chance of getting infections, a higher incidence of cancer, digestive side effects such as vomiting, nausea, or diarrhea, headaches, tremors, high blood pressure, high blood levels of cholesterol and triglycerides, and damage to the kidneys (https://www.niddk.nih.gov/health-information/diabetes/overview/insulin-medicines-treatments/pancreatic-islet-transplantation).

Despite the best efforts of numerous laboratories, it has not been possible to maintain human islet tissue isolated from cadaveric donors for more than several months ex vivo, and during that time no appreciable growth occurs and insulin synthesis and release progressively declines. Thus, cellular approaches to replace the insulin-producing beta cells have relied upon either immediate allogeneic transplantation following isolation from a cadaveric donor, or the use of iPSCs differentiated into insulin producing “beta cell-like” cells. Both of these approaches still require the use of immunosuppressant medications, and the iPSC approach harbors the additional long-term safety concerns discussed above.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

### Treatment of Type 1 Diabetes Via Ex Vivo Replication of Human Insulin-Producing Beta Cells.

It is well established that human islets containing beta cells maintained in tissue or organ culture do not propagate to therapeutically sufficient amounts to enable a reimplantation procedure. Thus, to maintain the cells in culture without continuous loss of cells requires a Viability Target to be identified and activated as described above. For beta cells, which are contained in the islets of Langerhans within the pancreas, it has been shown using mouse gene knockout experiments that Insulin Receptor Substrate-2 (IRS-2) is helpful for maintaining the viability (Hennige et al., 2003; Kuznetsova et al., 2016). Thus, animals in which both alleles of IRS-2 have been knocked out become diabetic shortly after birth, and transgene replacement of IRS-2 cures the diabetes. Therefore, IRS-2 fulfills the criteria of being a Viability Target (VT).

A compound that is known to activate IRS-2, designated HP-508, was utilized for these studies. HP-508 is a representative IRS-2 activator (Housey and Balash, 2018; Housey and Balash, 2023). HP-508 is classified as a viability target enhancer (VTE) as described herein.

An individual with T1D desires to have their remaining beta cell population expanded ex vivo in order to enable reimplantation of their own genetically identical tissue containing a sufficient number of beta cells to resolve their diabetes and normalize blood glucose levels. This individual will serve simultaneously as both donor and recipient, thus receiving their own, genetically identical beta cells upon completion of the procedure.

It has been shown that most T1D patients will have residual functional beta cells present in the pancreas. The presence of these functional beta cells can be demonstrated using a simple blood test to detect C-peptide which is a byproduct of endogenous insulin production by beta cells. To determine whether the patient has any residual beta cells remaining in the pancreas, a blood sample is drawn and c-peptide levels are determined. If the patient's c-peptide levels are at or above 5% of the normal range (˜0.04 ng/ml or higher), the patient has functional beta cells and is eligible for the procedure.

Next, a surgical biopsy is obtained from the pancreas. Generally, this will involve resection of the tail of the pancreas, obtaining approximately 20-25% of the total tissue. This is compatible with maintaining normal exocrine pancreatic function for the patient with the remaining 75-80% of pancreatic tissue. Depending upon the level of c-peptide production by the patient, smaller biopsy samples may be sufficient.

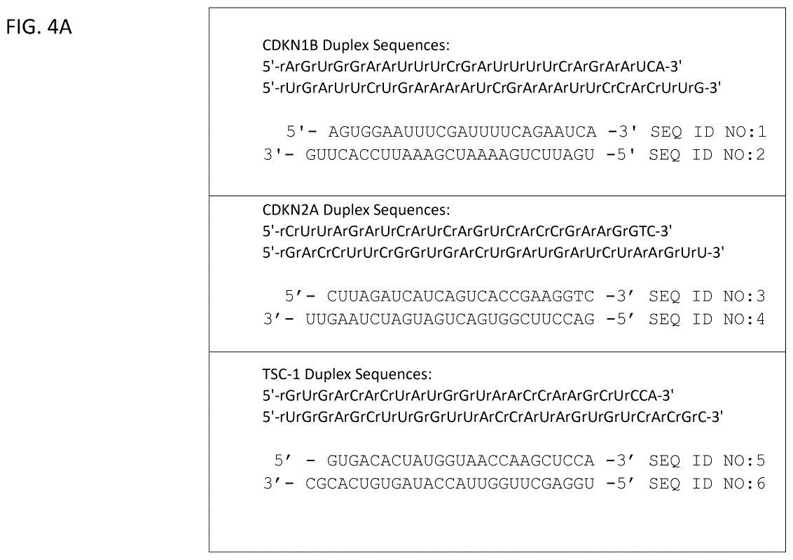

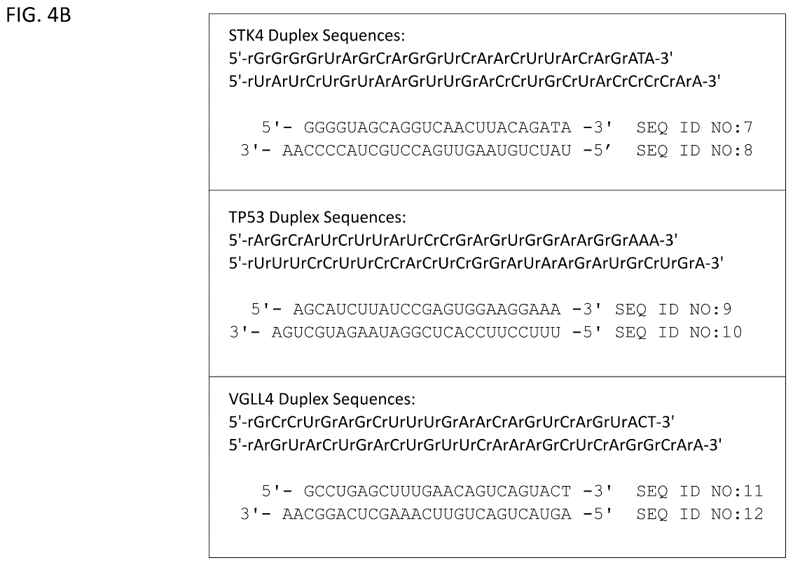

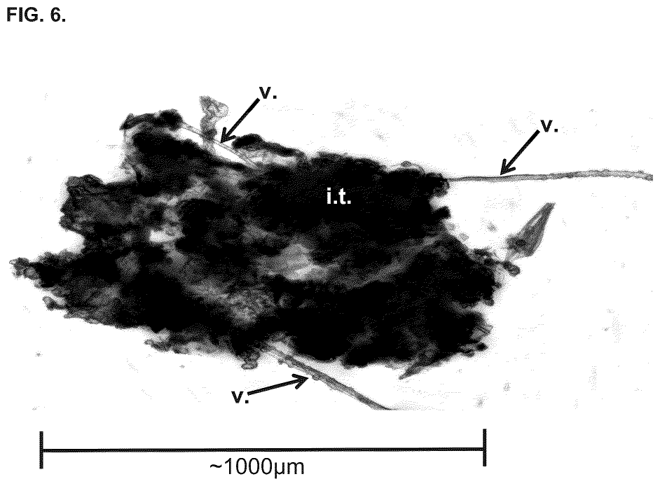

FIGS. 2-6 show the results of human islets that have been obtained by surgical resection from the pancreas of a cadaveric donor. Following resection, the pancreatic tissue is subjected to standard islet tissue isolation procedures as previously published for allogeneic donor islet transplantation procedures as well as autologous transplantation procedures for chronic pancreatitis (McEachron and Bellin, 2018).

Following islet isolation, the total number of islets isolated is determined using standard methods (Chetboun et al., 2023 and references therein). The islet issue is then expanded in culture conditions described as follows.

The islets isolated from the biopsy sample are first counted and the total number of islets at the outset are recorded. The tissue is then placed into tissue culture media that includes the addition of the IRS-2 activator HP-508. The tissue is then allowed to acclimate to the ex vivo culture conditions for a period of time, which may range from as little as one hour up to several days. The presence of HP-508 (a VTE for these purposes) serves to maintain the viability of the islet tissue while the routine passage of the tissue under specific conditions allows for the selective removal of fibroblast-like cells that are comingled with the biopsy specimen and divide at a much higher rate than human islet tissue. The ability of HP-508 to enhance cellular viability is shown in FIG. 2.

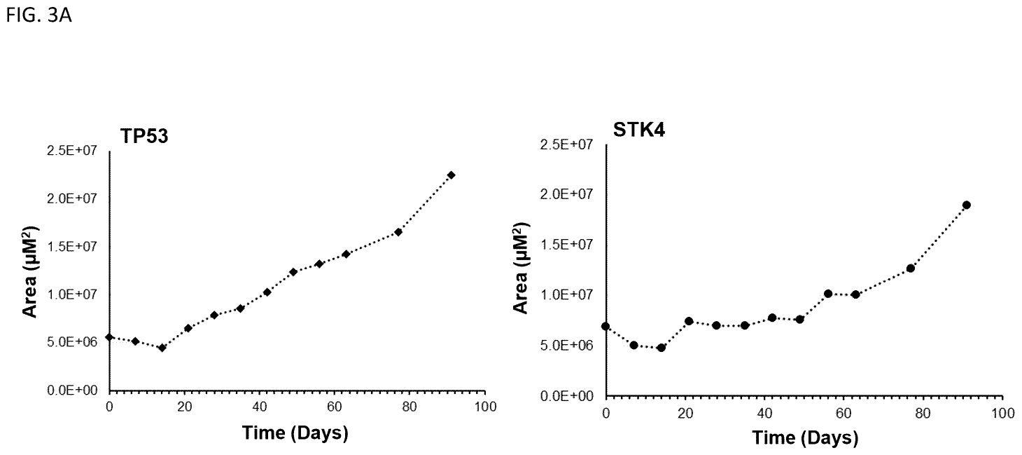

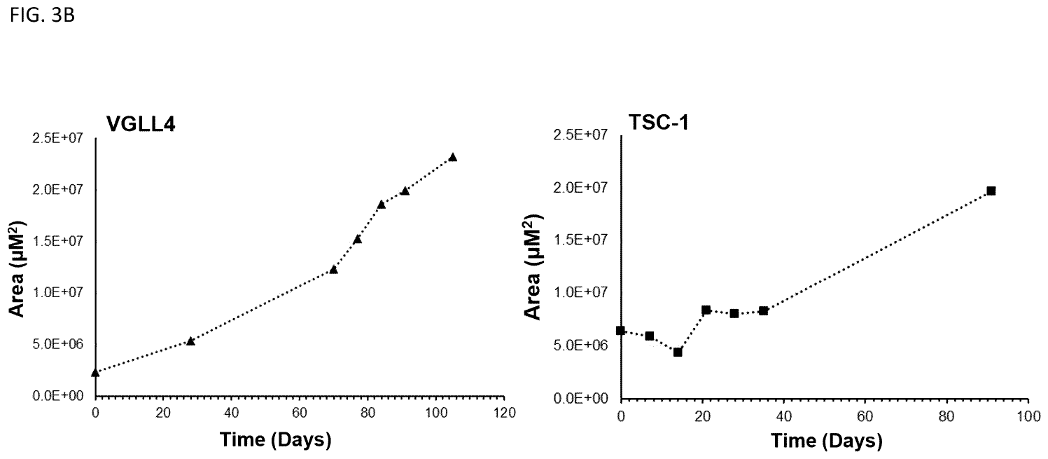

Islet tissue maintained in the presence of an IRS Activator remains viable for more than one year and exhibits a slow but sustainable growth pattern (FIG. 3). In our laboratories, human islet tissue has been maintained under such conditions for more than three years with continuous slow growth throughout, although, without addition of a cellular growth enhancer as described herein, the growth rate is insufficient to support a reimplantation procedure within a 12 month period.

After acclimation in culture is completed, a second substance is added to the culture medium that enhances cellular growth of the islet tissue. As shown in FIG. 3, after addition of a substance that enhances cellular growth rate of the islet tissue, the overall surface area of tissue increases by approximately 4-fold in a 64 day time period. (We have found that determination of tissue surface area provides a useful estimation of total wet tissue mass). This amounts to a tissue doubling time of approximately 32 days. While still being a slow division rate as compared to rapidly dividing cell lines that have been adapted to cell culture, this growth rate is sufficient to produce a total islet tissue mass equivalent to at least 400,000 islets within a six to seven month period of time starting with an initial islet mass of as few as 5,000 islet equivaents after biopsy. Typical allogeneic transplantations generally utilize between 5,000-10,000 islet equivalents per kg of body weight, which translates to approximately 350,000-700,000 islet equivalents per 70 kg individual (McEachron and Bellin, 2018). To the extent that the initial biopsy results in a larger number of islets at the outset, the minimum growth period would be reduced.

Substances that enhance cellular growth rate in the combined presence of an IRS Activator include those that activate proteins that regulate cell cycle division such as cyclin-dependent kinases, and substances that inhibit proteins that serve to block cell cycle division under normal circumstances. The latter include proteins that control metabolic state and proteins that prevent tumor formation (tumor suppressors).

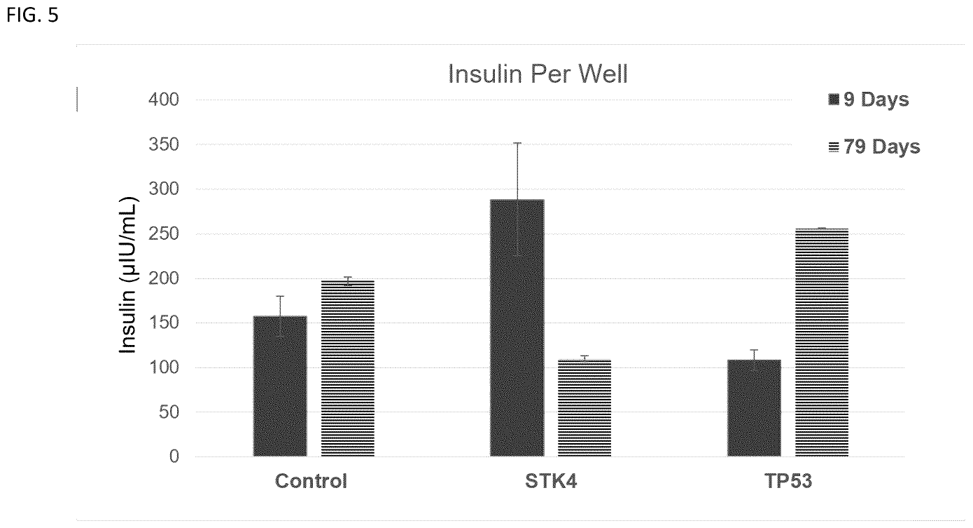

As shown in FIG. 5, the combination of an IRS Activator and a substance that enhances cellular growth leads to the formation of islet tissue masses of significant size that even develop their own vascular-like structures as they grow and enlarge.

The present disclosure renders obsolete the chronic use of immunosuppressive drugs, resulting in the avoidance of the plethora of side effects associated with their use as discussed above. Furthermore, unlike with iPSC-mediated approaches, a selected cell does not need to be dedifferentiated into an iPSC and then re-differentiated into an insulin-producing “beta-like” cell. This avoids the creation of hybrid cell types whose long-term stability after being placed back into the patient is uncertain.

### Example 2

Regeneration of Dopaminergic Neurons Isolated from Human Substantia Nigra Tissue

Parkinson's disease (PD) is characterized by loss of dopaminergic neurons located in the substantia nigra, a region located deep inside the human brain just above the basal ganglia. The dopaminergic neurons of the substantia nigra have also been shown to be dependent for viability on IRS-2 (Xie et al., 2021; Ramalingam et al, 2017). Loss of IRS2 function results in a reduction in dopaminergic neurons.

The compound HP-508 has been shown to activate IRS2. This compound acts as the VTE. Neurons are incubated in media with the amount of HP-508 necessary to maintain healthy cells.

Individuals with PD have a loss of dopaminergic neurons located within their substantia nigra. From a neurosurgical biopsy, a portion of healthy dopaminergic neurons is extracted from a patient and expanded ex vivo. At the time of the biopsy, an indwelling catheter is placed into the substantial nigra and is maintained for the duration of the neuronal growth and expansion period. The cells obtained are expanded ex vivo and the expanded cells are reimplanted later. Reimplantation of functional dopaminergic neurons helps ameliorate previous symptoms. In this example, as in Example 1, the individual is both the donor and recipient of the dopaminergic cells. These cells are therefore syngeneic, or autologous (genetically identical) to the surrounding tissue, thus reducing the possibility of autoimmune response from the patient.

After acclimation in culture is completed, a second substance is added to the culture medium that enhances cellular growth of the neurons. The proper amount of neurons necessary for reimplantation is determined by the physician based upon the degree of substantia nigra tissue loss observed in imaging studies. Furthermore, it has been shown that Brain-Derived Neurotrophic Factor (BDNF) is reduced in patients with PD. Thus, administration of a therapeutically effective amount of ex vivo expanded dopaminergic substantia nigra neurons will result in a return to normal blood levels of BDNF (Palasz et al., 2020). An enzyme linked immunosorbent assay utilizing a monoclonal antibody directed to an established antigen or fragment of BDNF can be performed using a blood sample to determine when sufficient numbers of cells have been reimplanted. Furthermore, the indwelling catheter may be maintained for a sufficient period of time to allow for additional cells to be reimplanted until such time as biomarkers associated with normal function are achieved.

### Example 3

### Treatment of Sensorineural Hearing Loss by Ex Vivo Expansion of Human Cochlear Hair Cells

Cells located in the cochlea of the inner ear are necessary for normal hearing. These cells harbor hair follicles and are essential cells for transmitting sound and vibration into electrical signals within neurons of the spiral ganglion that in turn transmit these signals to the to the brain. Sensorineural hearing loss is a major form of morbidity world-wide, for which hearing aids remain the mainstay of current treatment. It has not been possible to expand fully differentiated inner ear hair cells ex vivo, and stem cell approaches are being tested, but with same caveats as discussed herein (Qi et al, 2023).

Using the presently disclosed method, a biopsy of inner ear hair cells is obtained by an surgical otorhinolaryngologist and expanded ex vivo as described above in Examples 1 and 2. After ex vivo expansion, a therapeutically effective amount of the cells necessary to replenish the lost inner cochear hair cells are reinfused into the cochlea in sufficient numbers to repopulate the cochlear lining with functional hair cells. By this approach, hearing is restored without the need to resort to external hearing aids

### Summary

The skilled investigator will readily recognize the ability to adapt such an approach to other tissue reimplantation applications such as in Multiple Sclerosis, where non-regenerating oligodendrocyte and Schwann cell demyelination occurs, with specific motor neuron loss in spinal cord injuries, etc.) and other human disease where a progressive loss of a specific functional cell type has occurred.

### Citations

Blum B, Bar-Nur O, Golan-Lev T, Benvenisty N. The anti-apoptotic gene survivin contributes to teratoma formation by human embryonic stem cells. Nat Biotechnol. 2009 March; 27(3): 281-7.
Buzzard K, Chan W H, Kilpatrick T, Murray S. Multiple Sclerosis: Basic and Clinical. Adv Neurobiol. 2017; 15:211-252.
Chetboun M, Drumez E, Ballou C, Maanaoui M, Payne E, Barton F, Kerr-Conte J, Vantyghem M C, Piemonti L, Rickels M R, Labreuche J, Pattou F; Collaborative Islet Transplant Registry (CITR) Investigators study group. Association between primary graft function and 5-year outcomes of islet allogeneic transplantation in type 1 diabetes: a retrospective, multicentre, observational cohort study in 1210 patients from the Collaborative Islet Transplant Registry. Lancet Diabetes Endocrinol. 2023 June; 11(6): 391-401.
Cunningham J J, Ulbright T M, Pera M F, Looijenga L H. Lessons from human teratomas to guide development of safe stem cell therapies. Nat Biotechnol. 2012 September; 30(9): 849-57.
Fong C Y, Gauthaman K, Bongso A. Teratomas from pluripotent stem cells: A clinical hurdle. J Cell Biochem. 2010 Nov. 1; 111(4): 769-81.
Gholamzad M, Ebtekar M, Ardestani M S, Azimi M, Mahmodi Z, Mousavi M J, Aslani S. A comprehensive review on the treatment approaches of multiple sclerosis: currently and in the future. Inflamm Res. 2019 January; 68(1): 25-38.
Harris J P, Burrell J C, Struzyna L A, Chen H I, Serruya M D, Wolf J A, Duda J E, Cullen D K. Emerging regenerative medicine and tissue engineering strategies for Parkinson's disease. NPJ Parkinsons Dis. 2020 Jan. 8; 6:4.
Hennige A M, Burks D J, Ozcan U, Kulkarni R N, Ye J, Park S, Schubert M, Fisher T L, Dow M A, Leshan R, Zakaria M, Mossa-Basha M, White M F. Upregulation of insulin receptor substrate-2 in pancreatic beta cells prevents diabetes. J Clin Invest. 2003 November; 112(10): 1521-32.
Hinds P W, Weinberg R A. Tumor suppressor genes. Curr Opin Genet Dev. 1994 February; 4(1): 135-41.
Hirsch T, Rothoeft T, Teig N, Bauer J W, Pellegrini G, De Rosa L, Scaglione D, Reichelt J, Klausegger A, Kneisz D, Romano O, Secone Seconetti A, Contin R, Enzo E, Jurman I, Carulli S, Jacobsen F, Luecke T, Lehnhardt M, Fischer M, Kueckelhaus M, Quaglino D, Morgante M, Bicciato S, Bondanza S, De Luca M. Regeneration of the entire human epidermis using transgenic stem cells. Nature. 2017 Nov. 16; 551(7680): 327-332.
Housey G M, Balash M. Plant Extracts with Anti-Diabetic and Other Useful Activities. U.S. Pat. No. 11,633,446 B2. Ser. No. 16/612,545. Filed May 11, 2018. Issued Apr. 25, 2023
Housey G M, Balash M. Plant Extracts with Anti-Diabetic and Other Useful Activities. US Patent Application No. US 2023/0338449 A1. Ser. No. 18/214,183. Filed Jun. 26, 2023
Housey, G. M., Balash M. IRS Modulators. US Provisional Patent Application. Filed Oct. 4, 2023.
Housey G M, Balash M. Inhibition of Follistatin. US Patent Application No. US 20210207135 A1. Ser. No. 17/055,800. Filed May 17, 2019
Housey, G M, White, M F, Method of Screening Activators and/or Inhibitors of Insulin Receptor Substrate 2. U.S. Pat. No. 8,557,512 B2. Ser. No. 10/541,263. Filed Dec. 31, 2003. Issued Oct. 15, 2013
Jacque J M, Triques K, Stevenson M. Modulation of HIV-1 replication by RNA interference. Nature. 2002 Jul. 25; 418(6896): 435-8.
Kasper D, Hauser S, Jameson L, Fauci A, Longo D, Loscalzo, J. Harrison's Principles of Internal Medicine. McGraw-Hill Education; 19^{th} edition, Volume 2, 2015.
Kum J B, Ulbright T M, Williamson S R, Wang M, Zhang S, Foster R S, Grignon D J, Eble J N, Beck S D, Cheng L. Molecular genetic evidence supporting the origin of somatic-type malignancy and teratoma from the same progenitor cell. Am J Surg Pathol. 2012 December; 36(12): 1849-56.
Lee A S, Tang C, Cao F, Xie X, van der Bogt K, Hwang A, Connolly A J, Robbins R C, Wu J C. Effects of cell number on teratoma formation by human embryonic stem cells. Cell Cycle. 2009 Aug. 15; 8(16): 2608-12.
Kushner J A, MacDonald P E, Atkinson M A. Stem cells to insulin secreting cells: two steps forward and now a time to pause? Cell Stem Cell. 2014 Nov. 6; 15(5): 535-6.
Kuznetsova A, Yu Y, Hollister-Lock J, Opare-Addo L, Rozzo A, Sadagurski M, Norquay L, Reed J E, El Khattabi I, Bonner-Weir S, Weir G C, Sharma A, White M F. Trimeprazine increases IRS2 in human islets and promotes pancreatic β cell growth and function in mice. JCI Insight. 2016; 1(3): e80749.
Macleod K. Tumor Suppressor Genes. Curr Opin Genet Dev. 2000 February; 10(1): 81-93.
McEachron K R, Bellin M D. Total pancreatectomy and islet autotransplantion for chronic and recurrent acute pancreatitis. Curr Opin Gastroenterol. 2018 September; 34(5): 367-373.
Mutter G L. Teratoma genetics and stem cells: a review. Obstet Gynecol Surv. 1987 November; 42(11): 661-70.
Ohnishi K, Semi K, Yamamoto T, Shimizu M, Tanaka A, Mitsunaga K, Okita K, Osafune K, Arioka Y, Maeda T, Soejima H, Moriwaki H, Yamanaka S, Woltjen K, Yamada Y. Premature termination of reprogramming in vivo leads to cancer development through altered epigenetic regulation. Cell. 2014 Feb. 13; 156(4): 663-77.
Palasz E, Wysocka A, Gasiorowska A, Chalimoniuk M, Niewiadomski W, Niewiadomska G. BDNF as a Promising Therapeutic Agent in Parkinson's Disease. Int J Mol Sci. 2020 Feb. 10; 21(3): 1170.
Pancreatic Islet Transplantation|NIDDK (nih. gov) https://www.niddk.nih.gov/health-information/diabetes/overview/insulin-medicines-treatments/pancreatic-islet-transplantation
Payne S R, Kemp C J. Tumor suppressor genetics. Carcinogenesis. 2005 December; 26(12): 2031-45.
Prokhorova T A, Harkness L M, Frandsen U, Ditzel N, Schrøder H D, Burns J S, Kassem M. Teratoma formation by human embryonic stem cells is site dependent and enhanced by the presence of Matrigel. Stem Cells Dev. 2009 January-February; 18(1): 47-54.
Qi J, Huang W, Lu Y, Yang X, Zhou Y, Chen T, Wang X, Yu Y, Sun J Q, Chai R. Stem Cell-Based Hair Cell Regeneration and Therapy in the Inner Ear. Neurosci Bull. 2023 Oct. 3.
Ramalingam M, Kim S J. Protective effects of activated signaling pathways by insulin on C6 glial cell model of MPP+-induced Parkinson's disease. J Recept Signal Transduct Res. 2017 February; 37(1): 100-107.
Renewed Interest in Regenerative Medicine. EBioMedicine. 2018 January; 27:1-2.
Sender, R. and Milo, R. The distribution of cellular turnover in the human body. Nature Medicine volume 27, pages 45-48 (2021)
Sibley C R, Seow Y, Wood M J. Novel RNA-based strategies for therapeutic gene silencing. Mol Ther. 2010 March; 18(3): 466-76.
Sui G, Soohoo C, Affar el B, Gay F, Shi Y, Forrester W C, Shi Y. A DNA vector-based RNAi technology to suppress gene expression in mammalian cells. Proc Natl Acad Sci USA. 2002 Apr. 16; 99(8): 5515-20.
Takahashi K, Yamanaka S. Induction of pluripotent stem cells from mouse embryonic and adult fibroblast cultures by defined factors. Cell. 2006 Aug. 25; 126(4): 663-76.
Uchida T, Nakamura T, Hashimoto N, Matsuda T, Kotani K, Sakaue H, Kido Y, Hayashi Y, Nakayama K, White M F, Kasuga M. Deletion of Cdknlb ameliorates hyperglycemia by maintaining compensatory hyperinsulinemia in diabetic mice. Nature Medicine 2005 February; 11(2): 175-82.
Walton S P, Wu M, Gredell J A, Chan C. Designing highly active siRNAs for therapeutic applications. FEBS J. 2010 December; 277(23): 4806-13.
Withers D J, Gutierrez J S, Towery H, Burks D J, Ren J M, Previs S, Zhang Y, Bernal D, Pons S, Shulman G I, Bonner-Weir S, White M F. Disruption of IRS-2 causes type 2 diabetes in mice. Nature 1998; 391(6670): 900-4.
Withers D J, Burks D J, Towery H, Altamuro S, Flint C, White M F. Irs-2 coordinates Igf-1 receptor-mediated β-cell development and peripheral insulin signalling. Nature Genetics 1999; 23:32-40.
Xie Y, Zhang S, Lv Z, Long T, Luo Y, Li Z. SOX21-AS1 modulates neuronal injury of MMP+-treated SH-SY5Y cells via targeting miR-7-5p and inhibiting IRS2. Neurosci Lett. 2021 Feb. 16; 746:135602.
Yamanaka S. Pluripotent Stem Cell-Based Cell Therapy-Promise and Challenges. Cell Stem Cell. 2020 Oct. 1; 27(4): 523-531.
Yao T, Asayama Y. Animal-cell culture media: History, characteristics, and current issues. Reprod Med Biol. 2017 Mar. 21; 16(2): 99-117.
Yasuda S, Kusakawa S, Kuroda T, Miura T, Tano K, Takada N, Matsuyama S, Matsuyama A, Nasu M, Umezawa A, Hayakawa T, Tsutsumi H, Sato Y. Tumorigenicity-associated characteristics of human iPS cell lines. PLOS One. 2018 Oct. 4; 13(10): e0205022.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of expanding tissue or cells ex vivo which comprises:
(a) providing a biopsy of the tissue from an individual with a disease, wherein the disease has resulted in loss of the tissue;
(b) expanding the tissue or cells ex vivo to result in expanded tissue or cells, wherein the expanding comprises culturing the tissue in the presence of a compound that activates an Insulin Receptor Substrate (IRS) protein and a substance that enhances cell division; and
(c) reimplanting into the individual a therapeutically effective amount of the expanded tissue or cells.

2. The method of claim 1, wherein the disease comprises Type 1 Diabetes or Parkinson's disease.

3. The method of claim 1, wherein the substance comprises a siRNA that reduces expression of TP53, STK4, VGLL4, or TSC-1.

4. The method of claim 1, wherein the therapeutically effective amount of the expanded tissue comprises a mass equivalent to at least 400,000 cells.

5. The method of claim 1, wherein the individual is a human.

6. A method of treating a subject with diabetes which comprises:
(a) providing a biopsy of pancreatic tissue from an individual with diabetes;
(b) isolating islets of Langerhans contained in the biopsy;
(c) expanding the islets ex vivo to result in expanded islets, wherein the expanding comprises culturing the islets in the presence of a compound that activates an Insulin Receptor Substrate (IRS) protein and a substance that enhances cell division; and
(d) reimplanting into the individual a therapeutically effective amount of the expanded islets.

7. The method of claim 6, wherein the substance comprises a siRNA that reduces expression of TP53, STK4, VGLL4, or TSC-1.

8. The method of claim 6, wherein the therapeutically effective amount of the expanded islets comprises a mass equivalent to at least 400,000 islets.

9. The method of claim 6, wherein the reimplanting comprises portal vein infusion, omental injection, or a combination thereof.

10. The method of claim 6, wherein the method further comprises determining if blood glucose control is restored in the subject.

11. The method of claim 6, wherein the subject is a human.

12. A method of expanding human insulin-producing tissue ex vivo which comprises:
(a) providing a biopsy of pancreatic tissue from a subject with diabetes that has a relative or absolute deficiency of insulin;
(b) isolating islets of Langerhans contained in the biopsy;
(c) expanding the islets to result in expanded islets, wherein the expanding comprises culturing the tissue in the presence of a compound that activates an Insulin Receptor Substrate (IRS) protein and a substance that enhances cell division; and
(d) confirming that the expanded islets continue to produce insulin.

13. The method of claim 12, wherein the substance comprises a siRNA that reduces expression of TP53, STK4, VGLL4, or TSC-1.

14. The method of claim 12, wherein the therapeutically effective amount of the expanded islets comprises a mass equivalent to at least 400,000 islets.

15. The method of claim 12, wherein the method further comprises reimplanting the expanded islets.

16. The method of claim 15, wherein the reimplanting comprises portal vein infusion, omental injection, or a combination thereof.

17. The method of claim 12, wherein the biopsy is obtained from a human.

18-28. (canceled)
